(19) United States
(12) Patent Application Publication
(10) Pub. No.: US 2026/0155236 A1
(43) Pub. Date: Jun. 4, 2026

(54) TEXT DECODING METHOD FOR MEDICAL IMAGE FILE, AND ELECTRONIC DEVICE

(71) Applicant: Acer Incorporated, New Taipei City (TW)

(72) Inventor: Liang-Yu Ke, New Taipei City (TW)

(73) Assignee: Acer Incorporated, New Taipei City (TW)

(21) Appl. No.: 19/052,289

(22) Filed: Feb. 13, 2025

(30) Foreign Application Priority Data
Nov. 29, 2024 (TW) 113146413

(51) Int. Cl. G16H 30/20

(52) U.S. Cl. CPC G16H 30/20

(57) ABSTRACT

Provided are a text decoding method for a medical image file, and an electronic device. The text decoding method includes the following. A character set tag in a medical image file is read. Whether the character set tag is a predetermined tag value is determined. In response to the character set tag being the predetermined tag value, a current character coding standard is determined according to an operating system language of the electronic device. Text content of the medical image file is decoded according to the current character coding standard.

### CROSS-REFERENCE TO RELATED APPLICATION

This application claims the priority benefit of Taiwan application serial no. 113146413, filed on Nov. 29, 2024. The entirety of the above-mentioned patent application is hereby incorporated by reference herein and made a part of this specification.

### BACKGROUND

### Technical Field

The disclosure relates to a text decoding method for a medical image file, and particularly relates to a text decoding method for a medical image file, and an electronic device.

### Related Art

Artificial Intelligence (AI) medical technology is considered the key to the next generation of medical revolution. How to utilize AI to improve medical service quality and reduce the burden on doctors has become an important issue. Therefore, medical image files are used in more and more areas and receive increasing attention. Taking chest X-ray health examination as an example, this is a widespread and highly accessible medical examination. For the general public, this examination is highly available, and it is convenient and highly accurate to use AI technology to detect the bone density of the sternum and spine in X-ray image files and determine whether a patient has osteoporosis. The Digital Imaging and Communications in Medicine (DICOM) standard has emerged as a standardized medical image format and transmission protocol, which enables various medical imaging equipment and medical information systems from different manufacturers to be compatible with each other and to exchange data, so that medical institutions may seamlessly store, transmit, display, and share medical image information.

However, in terms of practical applications, to accommodate the compatibility of different equipment, the character coding methods used for the text data stored in DICOM files may not be unified in hospital systems. Due to the varying systems and character coding standards used by different hospitals, the text in DICOM files from many hospitals may not necessarily comply with UTF-8, which may easily lead to inconsistent coding standards between different hospitals. In such situations, decoding errors or garbled characters often occur when using software to read Chinese information from DICOM files, which may even affect the operation and accuracy of AI systems.

### SUMMARY

The disclosure provides a text decoding method for a medical image file, and an electronic device.

An embodiment of the disclosure provides a text decoding method for a medical image file, adapted for an electronic device, and the text decoding method includes the following. A character set tag in the medical image file is read. Whether the character set tag is a predetermined tag value is determined. In response to the character set tag being the predetermined tag value, a current character coding standard is determined according to an operating system language of the electronic device. Text content of the medical image file is decoded according to the current character coding standard.

An embodiment of the disclosure provides an electronic device, including a storage device and a processor. The storage device stores a program code. The processor is coupled to the storage device and accesses the program code to execute the following operations. A character set tag in a medical image file is read. Whether the character set tag is a predetermined tag value is determined. In response to the character set tag being the predetermined tag value, a current character coding standard is determined according to an operating system language of the electronic device. Text content of the medical image file is decoded according to the current character coding standard.

Based on the above, according to the embodiments of the disclosure, in the case where the character set tag in the medical image file is a predetermined tag value, the current character coding standard may be determined according to the operating system language of the electronic device, to decode the text content of the medical image file according to the current character coding standard. Accordingly, it is possible to prevent garbled characters or text decoding errors caused by inconsistency between the character set tag and the actually used character coding standard in the medical image file.

### BRIEF DESCRIPTION OF THE DRAWINGS

FIG. 1 is a schematic diagram illustrating the medical imaging system according to an embodiment of the disclosure.

FIG. 2 is a schematic diagram illustrating the electronic device according to an embodiment of the disclosure.

FIG. 3 is a flowchart illustrating the text decoding method for a medical image file according to an embodiment of the disclosure.

FIG. 4 is a flowchart illustrating the text decoding method for a medical image file according to an embodiment of the disclosure.

FIG. 5 is a flowchart illustrating the text decoding method for a medical image file according to an embodiment of the disclosure.

FIG. 6 is a flowchart illustrating the text decoding method for a medical image file according to an embodiment of the disclosure.

### DESCRIPTION OF THE EMBODIMENTS

Some embodiments of the disclosure will be described in detail below with reference to the accompanying drawings. In the following description, the same reference numerals shown in different drawings are considered as referring to the same or similar components. These embodiments are only part of the disclosure and do not disclose all possible implementations of the disclosure. More precisely, these embodiments are examples of methods and devices within the scope of the claims of the disclosure.

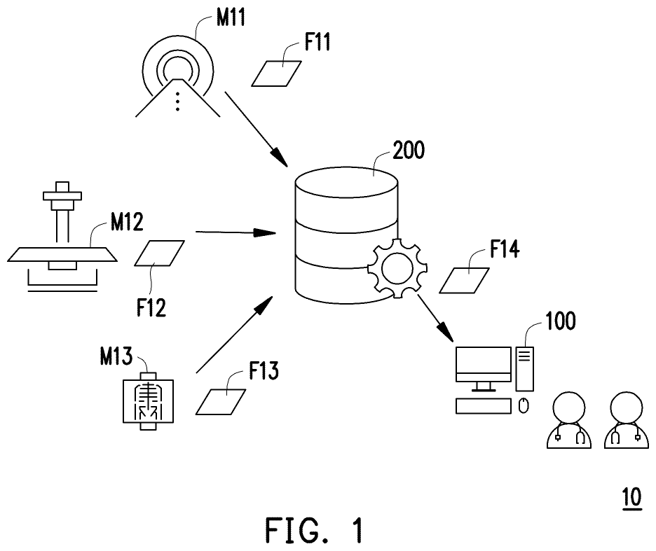

FIG. 1 is a schematic diagram illustrating the medical imaging system according to an embodiment of the disclosure. Referring to FIG. 1, a medical imaging system 10 includes multiple medical imaging devices M11 to M13, a medical image data server 200, and an electronic device 100.

The medical imaging devices M11 to M13 are configured to capture medical images. The medical images may be internal images of the human body, which are used to assist in diagnosing, treating, and monitoring various diseases. The medical imaging devices M11 to M13 may be, for example, X-ray equipment, Computed Tomography (CT) scanners, or Magnetic Resonance Imaging (MRI) machines, but the disclosure is not limited thereto. In other words, the medical images may be, for example, X-ray images, CT images, or MRI images.

The medical imaging devices M11 to M13 may convert the captured medical images into medical image files F11 to F13. The medical imaging devices M11 to M13 may store, transmit, and process the medical image files F11 to F13 in digital format. The medical image files F11 to F13 follow a specific medical image file format to facilitate sharing and analysis between different devices and systems. After the medical imaging devices M11 to M13 digitally convert the medical image data into the medical image files F11 to F13, medical professionals may use a medical imaging workstation (PACS system) for viewing, analysis, and diagnosis.

The medical image data server 200 may be a storage server in a Picture Archiving and Communication System (PACS). In some embodiments, the medical imaging devices M11 to M13 may transmit the medical image files F11 to F13 to the medical image data server 200 for the medical image data server 200 to store the medical image files F11 to F13.

In some embodiments, the PACS system may be integrated with the Digital Imaging and Communications in Medicine (DICOM) protocol, thereby ensuring that the medical images can be stored and transmitted in a standardized format to be compatible for the medical imaging devices M11 to M13 from different manufacturers. Specifically, the medical imaging devices M11 to M13 may transmit the medical image files F11 to F13 to the medical image data server 200 according to the DICOM protocol.

The electronic device 100 may be a computing device in the PACS system for displaying, processing, and analyzing medical images. For example, the electronic device 100 may be a medical imaging workstation or a remote computing device. In some embodiments, the electronic device 100 may access the medical image file F14 recorded by the medical image data server 200 according to the Hypertext Transfer Protocol (HTTP). The electronic device 100 may display the medical image and patient information in the medical image file F14 to a doctor. Alternatively, the electronic device 100 may perform artificial intelligence (AI) analysis based on the medical image and patient information in the medical image file F14 to generate an AI analysis result that assists in disease diagnosis.

It should be noted that whether to display the medical image and patient information in the medical image file F14 or to perform other processing based on the medical image and patient information in the medical image file F14, the electronic device 100 is required to perform a decoding operation on the text content in the medical image file F14 so as to convert the encoded text content in the medical image file F14 into a user-readable or system-recognizable text format.

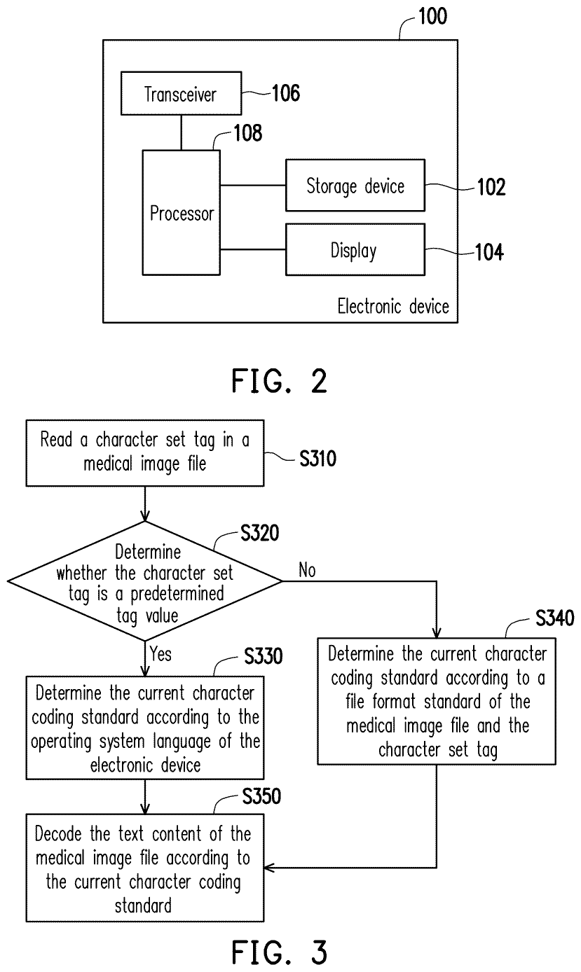

Referring to FIG. 2, FIG. 2 is a schematic diagram illustrating the electronic device according to an embodiment of the disclosure. In a different embodiment, the electronic device 100 may be various computer devices, smart devices, server devices, or combinations thereof, but the disclosure is not limited thereto. As shown in FIG. 2, the electronic device 100 may include a storage device 102, a display 104, a transceiver 106, and a processor 108.

The storage device 102 may be, for example, any type of fixed or removable Random Access Memory (RAM), Read-Only Memory (ROM), flash memory, hard disk, other similar devices, or combinations of these devices, which can be used to store multiple program codes or software modules.

The display 104 may be, for example, a Liquid Crystal Display (LCD), Light-Emitting Diode (LED) display, Organic Light-Emitting Diode (OLED) display, or other types of displays, but the disclosure is not limited thereto. The display 104 may be configured to display medical images.

The transceiver 106 transmits and receives data in a wireless or wired manner. The transceiver 106 may also execute operations such as low-noise amplification, impedance matching, mixing, up or down frequency conversion, filtering, amplification, and similar operations. The transceiver 106 may be configured to transmit and receive medical image files.

The processor 108 is coupled to the storage device 102, the display 104, and the transceiver 106, and may be a general-purpose processor, special-purpose processor, conventional processor, digital signal processor, multiple microprocessors, one or more microprocessors combined with digital signal processor cores, controller, microcontroller, Application Specific Integrated Circuit (ASIC), Field Programmable Gate Array (FPGA), any other type of integrated circuit, state machine, Advanced RISC Machine (ARM)-based processor, and the like.

In an embodiment of the disclosure, the processor 108 may access software modules, program codes, or instructions recorded in the storage device 102 to perform the text decoding method for a medical image file according to an embodiment of the disclosure, the details of which are described as follows.

Referring to FIG. 3, FIG. 3 is a flowchart illustrating the text decoding method for a medical image file according to an embodiment of the disclosure. The method of this embodiment may be performed by the electronic device 100 shown in FIG. 2. The details of each step in FIG. 3 will be described in conjunction with the components shown in FIG. 2.

First, in step S310, the processor 108 may read a character set tag in a medical image file. Specifically, the processor 108 may read metadata of the medical image file to read the character set tag from the metadata of the medical image file. The character set tag may also be referred to as a coding format tag. The character set tag is used to indicate the coding format of all text content in the medical image file, and the text content in the medical image file may include, for example, text content such as patient name, diagnostic result, image description, etc.

In some embodiments, the medical image file includes a Digital Imaging and Communications in Medicine (DICOM) file. The processor 108 may read Tag (0008,0005) in the DICOM file, which is the Specific Character Set tag defined by the DICOM standard.

In step S320, the processor 108 determines whether the character set tag is a predetermined tag value. Specifically, the processor 108 may determine whether the character set tag matches one or more predetermined tag values. In some embodiments, the medical image file may be a DICOM file, and the predetermined tag value may be “ISO_IR 6” or “ISO_IR 100”. In other words, the processor 108 may determine whether Tag (0008,0005) in the DICOM file is “ISO_IR 6” or “ISO_IR 100”.

It should be noted that since the actual character coding standard (for example, BIG5 standard) supported by the medical imaging equipment or medical systems may not be supported by the DICOM standard, the medical imaging equipment may set the character set tag to a predetermined value (for example, “ISO_IR 6” for Latin language) when generating the medical image file, while the text content in the medical image file is encoded using the actual character coding standard. As a result, a mismatch may occur between the actual character coding standard applied to the text content in the medical image file and the character coding standard indicated by the character set tag. Thus, according to the embodiment of the disclosure, the processor 108 may determine whether the character set tag in the medical image file is the predetermined tag value to determine how to perform a text decoding operation on the medical image file.

In step S330 (Yes in step S320), in response to the character set tag being the predetermined tag value, the processor 108 determines a current character coding standard according to an operating system language of the electronic device 100. In other words, in the case where the character set tag in the medical image file is the predetermined tag value, the processor 108 may determine the current character coding standard compatible with the actual coding method according to the operating system language of the electronic device 100, rather than directly determining the current character coding standard according to the character set tag in the medical image file. For example, the processor 108 may determine the current character coding standard according to the operating system language of the electronic device 100 by looking up a table. Alternatively, the processor 108 may determine whether to directly use a default character coding standard preset by the operating system according to the operating system language of the electronic device 100. Otherwise, the processor 108 may select an appropriate current character coding standard from multiple candidate character coding standards that support the operating system language of the electronic device 100.

In step S340 (No in step S320), in response to the character set tag not being the predetermined tag value, the processor 108 determines the current character coding standard according to a file format standard of the medical image file and the character set tag. For example, in the case where the processor 108 determines that the character set tag in the DICOM file is “ISO_IR 110” rather than “ISO_IR 6”, the processor 108 may directly determine the current character coding standard according to the DICOM standard and the character set tag “ISO_IR 110”. The coding mapping table of the DICOM standard records the correspondence relationship between multiple predefined tag values and multiple character coding standards. The coding mapping table of the DICOM standard can be referred to in the DICOM standard, which will not be elaborated here.

In step S350, the processor 108 decodes the text content of the medical image file according to the current character coding standard. Specifically, after determining the current character coding standard, the processor 108 may decode the text content of the medical image file according to a coding rule of the current character coding standard, thereby enabling the display 104 to correctly display the text content of the medical image file. The processor 108 may convert the hexadecimal numeric values generated through encoding in the medical image file into corresponding characters.

Since the current character coding standard used to decode the text content of the medical image file is determined according to the operating system language of the electronic device 100, the accuracy of text decoding for the medical image file can be improved.

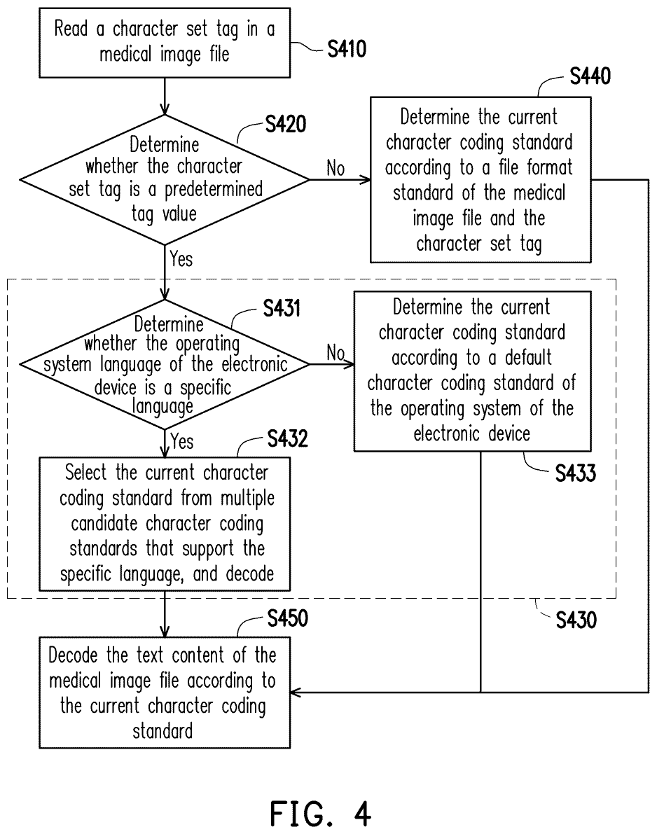

Referring to FIG. 4, FIG. 4 is a flowchart illustrating the text decoding method for a medical image file according to an embodiment of the disclosure. The method of this embodiment may be performed by the electronic device 100 shown in FIG. 2. The details of each step in FIG. 4 will be described in conjunction with the components shown in FIG. 2.

In step S410, the processor 108 may read a character set tag in a medical image file. In step S420, the processor 108 determines whether the character set tag is a predetermined tag value. The implementation details of the above steps S410 to S420 can be found in the previous embodiment, which will not be elaborated here.

In step S430, in response to the character set tag being the predetermined tag value, the processor 108 determines a current character coding standard according to an operating system language of the electronic device 100. In this embodiment, step S430 may be implemented as steps S431 to S433.

In step S431, the processor 108 determines whether the operating system language of the electronic device 100 is a specific language. The specific language may be set according to the country where the medical imaging equipment and electronic device are located. For example, the processor 108 may determine whether the operating system language of the electronic device 100 is Traditional Chinese.

In step S432 (No in step S431), in response to the operating system language being the specific language, the processor 108 selects the current character coding standard from multiple candidate character coding standards that support the specific language. In other words, in the case where the operating system language of the electronic device 100 is the specific language, the processor 108 may decode the text content in the medical image file according to the character set that supports the specific language. Furthermore, for Traditional Chinese with diverse characters, multiple candidate character coding standards may be suitable for text decoding. Therefore, the processor 108 may select one from multiple candidate character coding standards for decoding. The candidate character coding standards may include, for example, BIG5 standard, BIG5-2003 standard, BIG5E standard, BIG5-HKSCS standard, etc. It should be noted that since the BIG5 series standards also include ASCII character set, the processor 108 can correctly decode the text content even if all the text content in the medical image file is in English.

In step S433 (Yes in step S431), in response to the operating system language not being the specific language, the processor 108 determines the current character coding standard according to a default character coding standard of the operating system of the electronic device 100. In other words, in the case where the operating system language of the electronic device 100 is not the specific language, the processor 108 may directly select the default character coding standard preset by the operating system as the current character coding standard. For example, if the operating system language of the electronic device 100 is English, the processor 108 may directly use the UTF-8 (Unicode Transformation Format-8 bit) standard, which is the default of the operating system, as the current character coding standard.

In step S440, in response to the character set tag not being the predetermined tag value, the processor 108 determines the current character coding standard according to a file format standard of the medical image file and the character set tag. In step S450, the processor 108 decodes the text content of the medical image file according to the current character coding standard. The implementation details of the above steps S440 to S450 can be found in the previous embodiment, which will not be elaborated here.

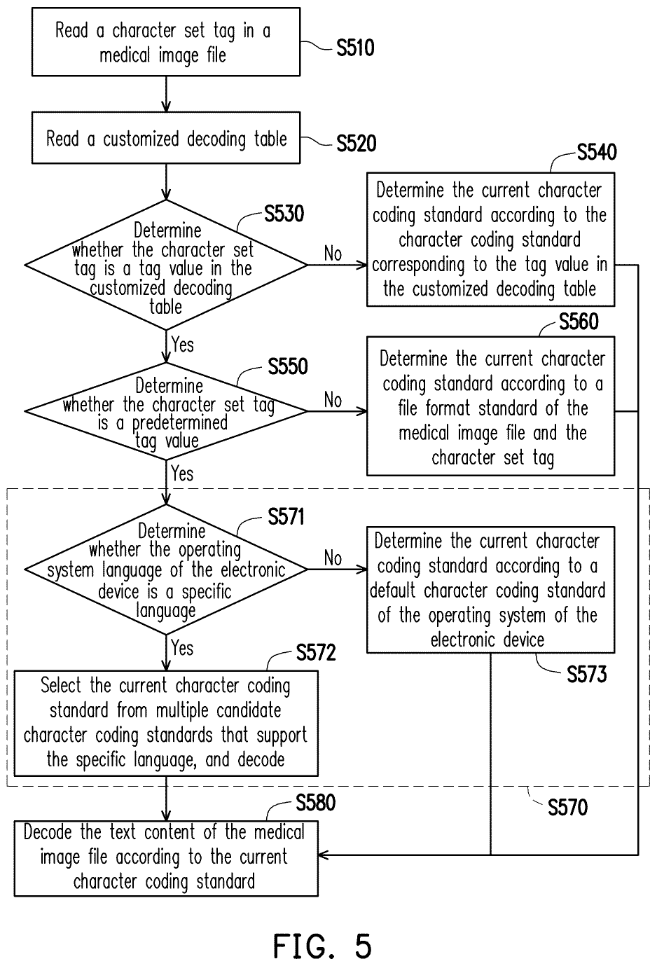

Referring to FIG. 5, FIG. 5 is a flowchart illustrating the text decoding method for a medical image file according to an embodiment of the disclosure. The method of this embodiment may be performed by the electronic device 100 shown in FIG. 2. The details of each step in FIG. 5 will be described in conjunction with the components shown in FIG. 2.

In step S510, the processor 108 may read a character set tag in a medical image file. Then, in step S520, the processor 108 may read a customized decoding table. This customized decoding table is a customized coding standard correspondence table, which may include multiple tag values and the corresponding character coding standards. In this embodiment, before referring to the coding standard correspondence table of the DICOM standard, the processor 108 may first refer to the customized decoding table. For example, Table 1 may be an example of the customized decoding table.

TABLE 1

| | |
|---|---|
| Tag value “ISO-IR X” | First character coding standard |
| Tag value “ISO-IR Y” | Second character coding standard |
| Tag value “ISO-IR Z” | Third character coding standard |

In step S530, the processor 108 may determine whether the character set tag is a tag value in the customized decoding table. For example, the processor 108 may determine whether the character set tag in the medical image file is “ISO-IR X”, “ISO-IR Y”, or “ISO-IR Z”.

In step S540 (Yes in step S560), in response to the character set tag being a tag value in the customized decoding table, the processor 108 determines the current character coding standard according to the character coding standard corresponding to the tag value in the customized decoding table. Referring to the customized decoding table in Table 1, in the case where the character set tag in the medical image file is “ISO-IR X”, the processor 108 directly selects the first character coding standard as the current character coding standard.

In step S550 (No in step S560), in response to the character set tag not being a tag value in the customized decoding table, the processor 108 determines whether the character set tag is a predetermined tag value. In step S560, in response to the character set tag not being the predetermined tag value, the processor 108 determines the current character coding standard according to a file format standard of the medical image file and the character set tag. In step S570, in response to the character set tag being the predetermined tag value, the processor 108 determines a current character coding standard according to the operating system language of the electronic device. In this embodiment, step S570 may be implemented as steps S571 to S573.

In step S571, the processor 108 determines whether the operating system language of the electronic device 100 is a specific language. In some embodiments, the character coding standard of the specific language may not be supported by the DICOM standard. In step S572 (No in step S571), in response to the operating system language being the specific language, the processor 108 selects the current character coding standard from multiple candidate character coding standards that support the specific language. In step S573 (Yes in step S571), in response to the operating system language not being the specific language, the processor 108 determines the current character coding standard according to a default character coding standard of the operating system of the electronic device 100.

In step S580, the processor 108 decodes the text content of the medical image file according to the current character coding standard. The implementation details of the above steps S550 to S580 can be found in the previous embodiment, which will not be elaborated here.

It is worth mentioning that, in some embodiments, in the case where the operating system language of the electronic device 100 is Traditional Chinese, the processor 108 may select one from multiple candidate character coding standards that support Traditional Chinese to determine the current character coding standard, as in steps S432 and S572 of the previous embodiment. The reason is that Traditional Chinese characters include rare or variant forms, and therefore the processor 108 may find the most suitable one from multiple candidate character coding standards based on the encoded hexadecimal data in the medical image file.

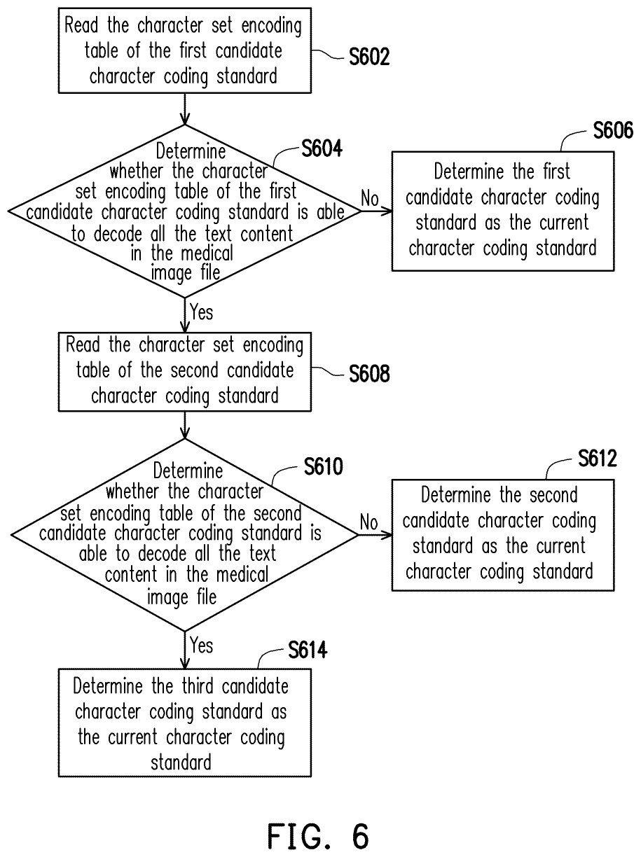

Referring to FIG. 6, FIG. 6 is a flowchart illustrating the text decoding method for a medical image file according to an embodiment of the disclosure. The aforementioned multiple candidate character coding standards may include a first candidate character coding standard, a second candidate character coding standard, and a third candidate character coding standard.

In step S602, the processor 108 reads the character set encoding table of the first candidate character coding standard. For example, the processor 108 may first read the character set encoding table (also referred to as character set) of the BIG5-2003 standard.

In step S604, the processor 108 determines whether the character set encoding table of the first candidate character coding standard is able to decode all the text content in the medical image file. For example, the processor 108 may determine whether all the hexadecimal numeric values generated through encoding in the medical image file can be found with corresponding characters in the character set encoding table of the BIG5-2003 standard. If the processor 108 cannot find characters corresponding to the hexadecimal numeric values generated through encoding in the medical image file in the character set encoding table of the BIG5-2003 standard, the processor 108 may determine that the character set encoding table of the first candidate character coding standard is unable to decode all the text content in the medical image file. Conversely, if the processor 108 can find characters corresponding to all the hexadecimal numeric values generated through encoding in the medical image file in the character set encoding table of the BIG5-2003 standard, the processor 108 may determine that the character set encoding table of the first candidate character coding standard is able to decode all the text content in the medical image file.

In step S606, in response to the character set encoding table of the first candidate character coding standard being able to decode all the text content in the medical image file, the processor 108 determines the first candidate character coding standard as the current character coding standard. In some embodiments, the processor 108 may convert all the hexadecimal numeric values generated through encoding in the medical image file into corresponding hexadecimal numeric values regulated by UTF-8 according to the current character coding standard (for example, the BIG5-2003 standard). Then, the processor 108 may successfully decode all the text content in the medical image file according to UTF-8.

In step S608, in response to the character set encoding table of the first candidate character coding standard being unable to decode all the text content in the medical image file, the processor 108 reads the character set encoding table of the second candidate character coding standard. In step S610, in response to the character set encoding table of the first candidate character coding standard being unable to decode all the text content in the medical image file, the processor 108 determines whether the character set encoding table of a second candidate character coding standard is able to decode all the text content in the medical image file. For example, the processor 108 may determine whether all the hexadecimal numeric values generated through encoding in the medical image file can be found with corresponding characters in the character set encoding table of the BIG5-HKSCS standard, to determine whether the character set encoding table of the second candidate character coding standard is able to decode all the text content in the medical image file.

In step S612, in response to the character set encoding table of the second candidate character coding standard being able to decode all the text content in the medical image file, the processor 108 determines the second candidate character coding standard as the current character coding standard. In some embodiments, the processor 108 may convert all the hexadecimal numeric values generated through encoding in the medical image file into corresponding hexadecimal numeric values regulated by UTF-8 according to the current character coding standard (for example, the BIG5-HKSCS standard). Then, the processor 108 may successfully decode all the text content in the medical image file according to UTF-8.

In step S614, in response to the character set encoding table of the second candidate character coding standard being unable to decode all the text content in the medical image file, the processor 108 determines a third candidate character coding standard as the current character coding standard. For example, in the case where the processor 108 determines that both the BIG5-2003 standard and the BIG5-HKSCS standard are unable to decode all the text content in the medical image file, the processor 108 forcibly uses the UTF-8 standard for decoding. In some embodiments, in the case where the processor 108 still cannot successfully decode through the UTF-8 standard, the processor 108 may report an exception error.

In addition, it should be noted that, in some embodiments, the processor 108 may determine whether the operating system language is Traditional Chinese. If the operating system language is Traditional Chinese, the processor 108 may initialize the character set encoding tables of multiple candidate character coding standards. In some embodiments, the processor 108 may determine whether a customized decoding table exists. If the customized decoding table exists, the processor 108 may determine whether the customized decoding table includes multiple candidate character coding standards. If the customized decoding table includes multiple candidate character coding standards, the processor 108 may initialize the character set encoding tables of multiple candidate character coding standards. In other words, the processor 108 may initialize the character set encoding tables that may be used according to actual needs.

In summary, according to the embodiments of the disclosure, in the case where the character set tag in the medical image file is a predetermined tag value, the current character coding standard may be determined according to the operating system language of the electronic device, to decode the text content of the medical image file according to the current character coding standard. Based on this, it is possible to prevent garbled characters or text decoding errors caused by inconsistency between the character set tag and the actually used character coding standard in the medical image file. In addition, a customized decoding table may be set in special circumstances, and an applicable character set encoding table may be selected for use according to the encoded encoding value, thereby ensuring that the text content in the medical image file can be correctly decoded.

Although the disclosure has been described with reference to the above embodiments, they are not intended to limit the disclosure. Those skilled in the art may make modifications and changes without departing from the spirit and scope of the disclosure. Therefore, the scope of protection of the disclosure shall be defined by the appended claims.

What is claimed is:

1. A text decoding method for a medical image file, adapted for an electronic device, the text decoding method comprising:
reading a character set tag in the medical image file;
determining whether the character set tag is a predetermined tag value;
in response to the character set tag being the predetermined tag value, determining a current character coding standard according to an operating system language of the electronic device; and
decoding text content of the medical image file according to the current character coding standard.

2. The text decoding method for the medical image file according to claim 1, further comprising:
in response to the character set tag not being the predetermined tag value, determining the current character coding standard according to a file format standard of the medical image file and the character set tag.

3. The text decoding method for the medical image file according to claim 1, wherein determining the current character coding standard according to the operating system language of the electronic device comprises:
determining whether the operating system language of the electronic device is a specific language; and
in response to the operating system language being the specific language, selecting the current character coding standard from a plurality of candidate character coding standards that support the specific language, and decoding.

4. The text decoding method for the medical image file according to claim 3, wherein determining the current character coding standard according to the operating system language of the electronic device further comprises:
in response to the operating system language not being the specific language, determining the current character coding standard according to a default character coding standard of an operating system of the electronic device.

5. The text decoding method for the medical image file according to claim 3, wherein in response to the operating system language being the specific language, selecting the current character coding standard from the plurality of candidate character coding standards that support the specific language, and decoding comprises:
determining whether a character set encoding table of a first candidate character coding standard is able to decode all the text content in the medical image file; and
in response to the character set encoding table of the first candidate character coding standard being able to decode all the text content in the medical image file, determining the first candidate character coding standard as the current character coding standard.

6. The text decoding method for the medical image file according to claim 5, wherein in response to the operating system language being the specific language, selecting the current character coding standard from the plurality of candidate character coding standards that support the specific language, and decoding further comprises:
in response to the character set encoding table of the first candidate character coding standard being unable to decode all the text content in the medical image file, determining whether a character set encoding table of a second candidate character coding standard is able to decode all the text content in the medical image file; and
in response to the character set encoding table of the second candidate character coding standard being able to decode all the text content in the medical image file, determining the second candidate character coding standard as the current character coding standard.

7. The text decoding method for the medical image file according to claim 6, wherein in response to the operating system language being the specific language, selecting the current character coding standard from the plurality of candidate character coding standards that support the specific language, and decoding further comprises:
in response to the character set encoding table of the second candidate character coding standard being unable to decode all the text content in the medical image file, determining a third candidate character coding standard as the current character coding standard.

8. The text decoding method for the medical image file according to claim 1, further comprising:
reading a customized decoding table;
determining whether the character set tag is a tag value in the customized decoding table; and
in response to the character set tag being the tag value in the customized decoding table, determining the current character coding standard according to a character coding standard corresponding to the tag value in the customized decoding table.

9. The text decoding method for the medical image file according to claim 8, wherein determining whether the character set tag is the predetermined tag value comprises:
in response to the character set tag not being the tag value in the customized decoding table, determining whether the character set tag is the predetermined tag value.

10. An electronic device, comprising:
a storage device storing a program code; and
a processor coupled to the storage device and configured to access the program code to execute:
reading a character set tag in a medical image file;
determining whether the character set tag is a predetermined tag value;
in response to the character set tag being the predetermined tag value, determining a current character coding standard according to an operating system language of the electronic device; and
decoding text content of the medical image file according to the current character coding standard.

11. The electronic device according to claim 10, wherein the processor is configured to execute:
in response to the character set tag not being the predetermined tag value, determining the current character coding standard according to a file format standard of the medical image file and the character set tag.

12. The electronic device according to claim 10, wherein the processor is configured to execute:
determining whether the operating system language of the electronic device is a specific language; and
in response to the operating system language being the specific language, selecting the current character coding standard from a plurality of candidate character coding standards that support the specific language, and decoding.

13. The electronic device according to claim 12, wherein the processor is configured to execute:
in response to the operating system language not being the specific language, determining the current character coding standard according to a default character coding standard of an operating system of the electronic device.

14. The electronic device according to claim 12, wherein the processor is configured to execute:
determining whether a character set encoding table of a first candidate character coding standard is able to decode all the text content in the medical image file; and
in response to the character set encoding table of the first candidate character coding standard being able to decode all the text content in the medical image file, determining the first candidate character coding standard as the current character coding standard.

15. The electronic device according to claim 14, wherein the processor is configured to execute:
in response to the character set encoding table of the first candidate character coding standard being unable to decode all the text content in the medical image file, determining whether a character set encoding table of a second candidate character coding standard is able to decode all the text content in the medical image file; and
in response to the character set encoding table of the second candidate character coding standard being able to decode all the text content in the medical image file, determining the second candidate character coding standard as the current character coding standard.

16. The electronic device according to claim 15, wherein the processor is configured to execute:
in response to the character set encoding table of the second candidate character coding standard being unable to decode all the text content in the medical image file, determining a third candidate character coding standard as the current character coding standard.

17. The electronic device according to claim 10, wherein the processor is configured to execute:
reading a customized decoding table;
determining whether the character set tag is a tag value in the customized decoding table; and
in response to the character set tag being the tag value in the customized decoding table, determining the current character coding standard according to a character coding standard corresponding to the tag value in the customized decoding table.

18. The electronic device according to claim 17, wherein the processor is configured to execute:
in response to the character set tag not being the tag value in the customized decoding table, determining whether the character set tag is the predetermined tag value.
